(19) United States
(12) Patent Application Publication
(10) Pub. No.: US 2007/0231827 A1
(43) Pub. Date: Oct. 4, 2007

(54) Method for Identifying Drug Targets by Mass Spectrometry

(76) Inventor: Xiaolong ZHANG, (US)

Correspondence Address: Hualin Zhong, 901HN, Hunter College, 695 Park Ave., New York, NY, 10021, US

(21) Appl. No.: 11/278,540

(22) Filed: Apr. 3, 2006

(51) Int. Cl. G01N 33/53; G01N 33/543

(52) U.S. Cl. 435/7.1; 436/518

(57) ABSTRACT

The present invention is an improved method for identifying in vivo protein targets, which bind specifically to a chemical compound. A sample is divided into two sets, to one of which a chasing compound is added. Two sets are then purified by affinity column, digested and labeled for mass spectrometry analysis. A comparison of the peak intensities of the same peptide between the two sets distinguishes a real binding target from a contamination.

### FIELD OF THE INVENTION

This invention relates to the analysis of biological matter and, more particularly, to identify in vivo proteins, which bind to a chemical compound, by affinity purification and mass spectroscopy.

### BACKGROUND OF THE INVENTION

In modern drug discovery, a protein, which is known to be involved in a disease, is often used as a drug target in a high-throughput kinetic assay to search for its inhibitors. Lead compounds identified from the high throughput screening (HTS) are then subject to further validation, optimization and development steps before becoming drug candidates.

With the vast advance in screening technologies over the last decade, it became relatively easy to identify a set of lead compounds for a given drug target. However, due to high cost and high failure rate in the drug development process, it is crucial to make a go/no-go decision about whether to put a lead compound into a development pipeline. This decision making process requires various information about the lead compound, such as its physical and chemical properties, its adsorption, its metabolism, and its binding targets in vivo. Two questions are often asked regarding a lead compound's in vivo targets: 1) whether the protein target used in the HTS is the real target for the lead compound in vivo? 2) Whether other proteins can also interact in vivo with the lead compound? Answers to these two questions are essential to predict the efficiency and toxicity of the lead compound as a drug candidate.

A conventional approach to identify the in vivo protein targets of a compound is the affinity chromatography technique, where proteins binding to the compound are purified with an affinity matrix on which surface the compound molecule or its analogs are immobilized as ligands. For example, in 1986, Harding and others reported the identification of two 17 KD proteins which bound to an immunosuppressive drug cyclosporin A and named them cyclophilin (JBC, Vol. 261, 8547-8555, 1986). In 1991, Liu and others reported identification of a protein phosphatase named calcineurin as a common target for both cyclosporin A and FK5506, two widely used immuno-suppressor drugs (Cell, Vol. 66, 807-815, 1991). There are many other examples that in vivo drug targets were isolated by similar affinity purification technique using drugs or their analogs as ligands, followed by identification of the affinity purified proteins by chemical sequencing (Edman sequencing) or mass spectrometry based peptide sequencing (JBC Vol. 267, 5503-5507, 1992; PNAS Vol. 94, 6110-6115, 1997; PNAS, Vol. 100, 15434-15439, 2003).

However, there are some intrinsic problems and limitations in using the affinity purification approach to identify in vivo drug targets. First, the binding affinity of an immobilized drug ligand to its target proteins may not be high enough to allow specific purification of these target proteins. Second, since the concentrations of proteins inside cells may vary over 7-13 orders of magnitudes, even with a high affinity ligand, it is still very difficult or even impossible to purify low-abundant target proteins without contamination of some high-abundant cellular proteins. Third, for a given affinity purification, the surface area covered by ligands accounts only for very small percentage of total surface area that contacts with protein samples. So inevitably, many contaminant proteins will be retained by affinity column due to their specific or non-specific interaction with matrix itself instead of ligands. For these reasons, it is often not surprising to find that elution from affinity purification contains many different proteins (sometimes hundreds or even thousands of different proteins). Consequently, it is very difficult to identify the real protein targets of the drug compound from a large amount of contaminations.

Several approaches have been used to overcome the problems describe above. The most widely used approach is to include a negative control for affinity purification. The negative control is a sample purified in the way identical to the experimental ones except that the matrix used does not incorporate the specific ligand. The proteins eluted from the affinity matrix and that from the control matrix are separated and compared side by side in a SDS-PAGE gel. Only the protein bands that are present in the “positive” sample but absent in the control sample are considered to be potential drug targets (JBC PNAS Vol. 94, 610-615, 1997). Although such control is helpful to improve the specificity, it still has some limitations: 1) since the column used in experimental sample is different from the one in the negative control, the surface properties of the two columns are different. Therefore, a differential amount of an eluted protein between the two columns may not be necessarily and entirely resulted from the specific ligand. Such matrix effect may lead to the appearance of a large number of “false positives.” Since functional study of each candidate protein often require much time and effort, a large number of “false positives” make it very difficult to identify the real target proteins; 2) The concentration of the target proteins may be too low to be a distinct band on a gel, or the target proteins may co-migrate with other non-specific proteins. In either case, the method, which identifies specific drug target proteins on the basis of differential protein band patterns on a gel, may miss the real target proteins and lead to a “false negatives.” Some other approaches have also been used to overcome the specificity problem, such as using 2-D electrophoresis instead of SDS-PAGE to increase resolution of protein elute, but it still suffers similar limitations described above. (PNAS, Vol. 100, 15434-15439, 2003).

In recent year, mass spectrometry has been coupled with affinity purification in drug target identification. In U.S. Pat. No. 6,864,099, after affinity purification, two protein samples attached with different isotopes are mixed together for mass spectrometry analysis in order to determine the difference of proteins present in the two samples. In U.S. Pat. Nos. 6,391,649 and 6,642,059, cell extracts are prepared from two sets of cells cultured with different isotope nutrient. The difference of proteins present in the cell extracts are determined by mass spectrometry. In U.S. Pat. No. 6,379,970, a method is described to distinguish different samples with different labeling fluorescent group. Nevertheless, these methods compared two different cell extracts to determine a difference of proteins between them. Since it is impossible to have two cell extracts identical, no matter how carefully they are prepared, it is unavoidable to have “false positives” and “false negatives.”

It is, therefore, the primary object of the present invention to provide an improved method for drug target identification, which offers higher specificity in determining true targets than the methods in prior arts. Higher specificity refers to both lower “false positives” and lower “false negatives.”

Another object of the present invention is to eliminate variation of initial protein samples.

Another object of the present invention is to provide a method for drug target identification, which can determine quantitatively the binding affinity of a target protein to its ligand.

A further object of the invention is to provide a method for drug target identification, which can be automatable.

### SUMMARY OF THE INVENTION

In the attainment of the foregoing and other objects and advantages of the present invention, an important feature resides in presenting free ligands in at least one set of samples at the affinity purification step.

Another important feature resides in using same affinity matrix for all sets of samples at the affinity purification step.

Another important feature resides in using at least two different isotope labeling to distinguish different set of samples.

Another important feature resides in using mass spectrometry to distinguish each individual protein present in samples and further determine their identities.

In the first preferred embodiment of the invention chosen for illustration, a protein sample is divided into at least two sets, at least one of which is mixed with certain amount of free ligands. The free ligands are either identical to or analogs of the ones immobilized on a matrix used at the following affinity purification step. Both sets go through the affinity purification step separately. Elution of each set from the purification step is first subject to proteolytic enzyme digestion, and then labeled with an isotope. At least two isotopes are required in order to distinguish proteins in different sets of elution. After labeling, a portion from sets labeled with different isotope are mixed together and applied to MASS analysis.

The second preferred embodiment of the invention is almost identical to the first one, except that elution from the affinity purification step is first subject to isotopic labeling, and then digested with proteolytic enzymes.

The third preferred embodiment of the invention is almost identical to the first one, except that elution from the affinity purification step is subject to isotopic labeling and proteolytic enzyme digestion at the same time.

In another preferred embodiment of the invention chosen for illustration, the invention could be used to compare the binding affinity of different ligands to commonly shared target proteins. A protein sample is divided into two sets; each one of them is mixed with different free ligand. Both sets go through the affinity purification step separately. Elution of each set from the purification step is first subject to proteolytic enzyme digestion, and then labeled with an isotope. At least two isotopes are required in order to distinguish proteins in different sets of elution. After labeling, a portion from sets labeled with different isotope are mixed together and applied to MASS analysis.

### BRIEF DESCRIPTION OF THE DRAWINGS

Other objects and advantages of the invention will become apparent from the detailed description thereof contained hereinbelow, taken in conjunction with the drawings, in which:

FIG. 1 illustrates the procedure for identifying in vivo protein targets of a lead compound.

FIG. 2 illustrates the principle of the invention.

FIG. 3 is a sample of SDS-PAGE of an affinity purified example and a control example.

FIG. 4 is an example of MS/MS spectrum and its assignment.

FIG. 5 is an example of searching result based on mass spectrometry data.

FIG. 6 illustrates the principle of the invention in comparing the binding affinity of different ligands to commonly shared target proteins.

### DETAILED DESCRIPTION OF THE INVENTION

In this invention, drug is a synonym of medicine, a chemical or biological compound used to treat or cure human, animal, or plant disease.

Affinity Chromatography

The invention described here is based on affinity chromatographic technique, which is able to purify or enrich proteins that bound to an immobilized drug-like molecule in order to identify the in vivo target of the drug. A successful separation requires that the drug or its derivative is covalently attached to some type of bed material, which is called matrix, and that the immobilized ligand still retain its affinity and activity towards the target protein. There are several key factors that need to be considered in developing a drug affinity purification strategy, includes 1) How to select a drug-like ligand; 2) How to select a matrix; 3) What kind of chemical reaction should be used to couple the selected ligand to the selected matrix; 4) What kind of protein extract should be used for purifying the target proteins.

Ligand

Several factors need to be considered in selecting a ligand for affinity purification of the target proteins. First, it should contain a reactive chemical group that can be easily and specifically coupled to an activated matrix. In many cases, a real drug candidate may not contain such a chemically reactive group, since chemically reactive groups may become easy targets during in vivo drug metabolism leading to inactivation of the drug molecules or cause some toxic effect. So, it is often necessary to pick a ligand that has similar chemical structure to the drug or a drug candidate, but with some additional chemically reactive group. Second, if such a drug derivative is used as a ligand, the ligand should have similar binding and inhibition profile as the drug, and the ligand should have similar binding and inhibition profile to the free-drug after such a ligand is immobilized on a matrix. Therefore, the chemical group attached to a matrix should be opposite to the target-binding group. The structure information about the binding of the drug to the protein target will be very important in designing a good ligand for affinity purification. However, there are some case that protein binding to the sites different from that occupied by the intended drug target is in question, it will be necessary to explore different way to attach a ligand to a matrix. Third, since some chemical derivatization may be needed to make an active and matrix attachable ligand, and each step in a chemical synthesis may cost significantly, the economic factor should be considered in selecting a ligand.

Matrix

Comparing to selecting a good ligand, it is often much easier to select a matrix, since there are many different types of matrix that are commercially available and they can accommodate many different coupling methods. Since the choices for matrix are very large, we will only discuss a few commonly used matrix material here. A spacer arm is often used to connect small molecular ligands to matrix to reduce steric interference. Activated Matrix gels with different length of spacer arm are also available from various sources.

1) Agarose and Cross-Linked Agarose

Bead formed agarose gels has been used widely as matrix for affinity chromatography (e.g. Sepharose CL-6B from Pharmacia). Agarose based beads have some excellent profile as a matrix. The building blocks of agarose, D-galactose and 3-anhydrogalactose, provide an uncharged hydrophilic matrix with very abundant primary and secondary hydroxyl groups, which can be activated for ligand coupling. The cross-link agarose can further extend the application of beaded agarose gels in affinity purification. The cross-linking is often introduced by reagents such as epichlorhydrin or divinyl sulfone (DVS). The resulted agarose beads are more stable chemically and mechanically, while their porosity have not been reduced significantly by cross-linking. For example, cross-linked agarose beads Sepharose CL-6b can tolerate organic solvents, high temperature (e.g. for autoclaving) and disruptive eluents such as guanidine hydrochloride.

2) Sephacryl

Sephacryl is another type of matrix material developed by Pharmacia and is commercially available. Sephacryl is a copolymer produced by polymerizing allyl dextran with cross-linking monomer N, N′-methylene-bis(acrylamide). The resulting polymer contains linear chains of polymeric glucose molecules held together by bis(acrylamide) induced cross-linking. The matrix based on sephacryl polymer is stable under a wide range of experimental conditions such as high and low pH, detergents and organic solvents commonly used in chromatography. Since the pore size of this type of polymers can be controlled by degree of cross-linking, products with a range of pore sizes are offered commercially. For example, Sephacryl S-400 have exclusion limit of 8 million Daltons, which should be able to accommodate the bind of large proteins and protein complexes. Since sephacryl is a polymerized dextran, it contains abundant hydroxyl groups that can be activated for coupling of affinity ligands. The activation methods include CDI, CNBR, epoxy, which are described in coupling chemistry section.

3) Polystyrene and its Derivatives

This type of matrix was developed by Applied Biosystem with trade name “Poros”. The basic backbone of this type of matrix is the polystyrene/divinylbenzene copolymer. It was suggested that this type matrix contains two different types of pores: larger size pores (600-800 nm), called “through pores”, which can allow convective flow through the beads, and smaller size pores (50-100 nm), called “diffusive pores”, which can allow diffusion of mobile phase into them. As a result, the transport of solute into the matrix can occur at much high speed, and the linear flow rate of a column can be significantly increased. Also, due to the physical and chemical properties of polystyrene, this type media is very robust and stable even under some extreme conditions, such as very high or very low pH (pH1-14), high pressure (3000 psi) and high temperature. So the poros media is very suitable for HPLC application. Since the polystyrene backbone itself is difficult to be activated for coupling of ligands, the coupling of ligands to poros matrix is via the cross-linked coating of polyethyleneimine or polyhydroxylic polymers.

4) Ultrogel and Magnogel

The trade name Ultragel represents a line of products from IBF. The Ultragel AcA gels are the mixture of agarose and polyacrylamide. It is suggested that such a matrix may offer the advantage of both agarose-based matrix (such as mechanical stability) and polyacrylamide based matrix (such as lack of interference from carbohydrate). The pore size of this type of matrix can be controlled by varying the concentration of polyacrylamide and agarose, and their exclusion limit can be as high as 3 million Daltons. One particular product in this product line is called Magnogel AcA44, which contains iron oxide (Fe3O4) particles in the core of the bead. Such magnetic beads can be useful in purifying binding protein. Similar to sephacryl type of matrix, the agarose components in Ultragel beads can be activated by CNBr, DVS, or epoxy method for the coupling of lignads. More detailed information about this matrix material can be found in: Ultrogel and Magnogel, Practical Guide for Use in Affinity Chromatography, 1979, Pharmindustrie, Clinchy, France.

Chemistry of Coupling Ligands to Matrix

In affinity chromatography, the typical way to couple a ligand to a matrix is first to activate the matrix, based on the chemically reactive group a ligand contains, and then couple the active group in the ligand to the activated matrix. There are many types of reactive groups in affinity ligands that can be used for immobilization. The most widely used groups are primary amine (—NH2), sulphydryl (—SH), hydroxyl (—OH), carbonyl (—CHO) and carboxyl. The coupling reaction involved in these active groups are often efficient and specific, and many type of pre-activated matrix materials are commercially available from Vendors such as Pharmacia, Pierce, Bio-Rad so a ligand can be coupled to a selected matrix in a simple and one-step reaction. The most common matrix activation methods are described briefly in the following:

1) Primary Amine Containing Ligands

For a primary amine containing ligand, the methods for matrix activation includes cyanogen bromide (CNBr), N-hydroxy succinimide esters (NHS) and carbonyl diimidazole (CDI). CNBr and NHS are used to activate hydroxyl group, which is very abundant in many type of matrix such as Sepharose. CDI can activate both hydroxyl and carboxyl-groups.

2) Sulphydryl Containing Ligands

For a sulphydryl containing ligand, iodoacetyl and pyridyl disulfide are two most common methods used for matrix activation. Both activation methods are very selective and efficient, and the activated matrix can be coupled with a sulphydryl containing ligand rapidly at pH8.0-8.5.

3) Hydroxyl Containing Ligands

For a hydroxyl containing ligand, bisoxiranes (epoxy) is the most common used activation method. The epoxy-activated matrix can also react with primary and second amine, sulphydryl, in addition to hydroxyl group. At neutral pH, sulphydryl group reacts with epoxy-activated matrix more rapid than amine group. Coupling with hydroxyl group requires much higher pH (pH11-12) and a reaction may take as long as 48 hours. One special feature of the epoxy activation method is that the linkage formed between matrix and a ligand is much more stable than most of other methods. This is useful in the case that ligand leak is a problem.

4) Carbonyl-Containing Ligands

For a carbonyl-containing ligand, hydrazide is the method of choice for matrix activation. For example, hydroxyl groups in a matrix can be oxidized with sodium periodate to generate an aldehyde-containing matrix. The intermediate is then react with adipic dihydrazide to produce the active hydrazide-containing support. The hydrazide-activated matrix can then be used to couple carbonyl-containing ligands.

5) Carboxyl-Containing Ligands

For a carboxyl-containing ligand, although there is no direct activation method to prepare a matrix that can spontaneously react with carboxyl group, cross-linkers named carbodiimides (e.g. EDC: CH₃—CH₂N═C═N—CH₂CH₂CH₂NH(CH₃)₂, Pierce, Cat No. 22980) can be used to couple carboxyl to primary amines to form an amide bond, or couple carboxyl to hydrazide to form hydrazone bond (Bayer et al, 1987, Anal. Biochem. Vol. 161, 123).

Protein Extract

The protein extract used for affinity purification of drug targets should prepare according to biology of the target proteins in study. The protein extract may be prepared from tissue, primary cell, or a cell line. Some fractionations of proteins extract may be very helpful to enrich target proteins. For example, if membrane proteins are of interest, use membrane protein fraction will significantly reduce contaminant proteins and increase the chance to purify the target proteins by affinity chromatography. Due to the complexity of protein purification, there is no best procedure that can fit different samples. A protocol for preparing a protein extract has to be developed experimentally.

Stable Isotopic Labeling for Relative Protein Quantification

The invention uses two different stable isotope tags to differentially label the eluted protein pools from positive and control samples, because such a differential labeling allows the detecting of protein differential between these two samples by mass spectrometry.

Although stable isotopic labeling technique has been used for many years in many fields, the recent emerging field of proteomics, as a way to study proteins in a whole cell, has renewed the interest in isotopic labeling technique (Mann, Nature Biotechnology Vol. 17, 1999). The basic principal of relative quantification of protein by stable isotope labeling is as following: In order to determine the relative ratio of one protein between two samples, namely sample A and sample B, one can label all proteins in sample A with a specific chemical tag T1, and label all proteins in sample B with the chemical tag T2, while T1 and T2 have identical chemical structure except that T1 contains natural isotope and T2 contains at least some different stable isotopes (usually heavy isotopes such as deuterium, carbon C-13, oxygen O-18, etc). As a result, there will a certain mass difference between a protein/peptide tagged T1 and that tagged with T2, which can be measure be mass spectrometric techniques. Since a T1 tagged protein/peptide is identical to T2 tagged same protein/peptide in term their chemical structure, the ratio of MS signal of a T1 tagged protein/peptide to that of T2 tagged peptide/protein will be equal to the ratio of their concentration between sample A and sample B.

Many different methods have been developed for stable isotopic labeling. These methods differ in the amino acid residues that a stable isotopic tag is attached, as well as mechanism of the coupling reactions. Some of the methods are described in the following.

1) ICAT Method.

ICAT stands for isotopic coded affinity tag. The method was developed in late 1990s by R. Aebersold et al (Gygi. Et al. Nature Biotechnology, 17:994-999, 1999). The labeling reagent contains a biotin, a thiol-specific reactive group and a linker that are ether in heavy form (contain 8 deuterium) or in light (hydrogen) form. The reagents can react specifically with Cys residues in a protein. After proteolysis of proteins, the labeled peptides can be isolated by using biotin-affinity technique. Since Cys is a rare amino acid in a protein, and only about 10% of total peptides from a trypsin digestion of a protein are Cys-containing peptides, the complexity of the samples containing multiple protein digests can be reduced. The labeling reagents are now commercially available from Applied Biosystem.

2) N-Terminal Labeling by H4/D4 Nic-NHS

The method was developed by Peter James et al (Munchbeach. et al, Anal. Chem. 72:4047-57, 2000). The labeling reagents are 1-([D4/H4]-nicotinoyloxy) succinimide, which can react specifically with N-terminal amine of peptides and Lys residues. To avoid the side-reaction with Lys side chain, proteins was first react with succinic anhydride to allow succinylation of virtually every Lys residues in a protein. The proteins are digested, and the N-terminals of resulted peptides are labeled ether with heavy or light tags. This method can also be for the quantification of protein modifications.

3) N-Terminal Isotopic Tagged (NIT)

Similar to the Nic-NHS method, the NIT method can also specifically and differentially label each peptide N-terminals (Rapid communication in mass spectrometry, 16, 2325-2332, 2002). In this method, proteins are first digested with a proteolytic enzyme such as trypsin. Then the Lysine residues in the resulted peptides are converted to homoarginine by reacting with o-methyl isourea. This conversion can prevent the acylation of Lys in the next step, and also increase the mass spectrometric signal of Lys containing peptides. Then, the N-terminal of peptides was labeled by reaction with DH5-proponic anhydride of D5-propronic anhydride.

4) O16/O18 Labeling

Different from above labeling methods, this method can differentially label peptides simultaneously during a proteolytic digestion. For example, protein digestion in buffer with O18-water can introduce 2 O18 atoms to peptide C-terminals, (Rapid communication in mass spectrum 14, 1226-1232, 2000; Anal. Chem. Vol. 73, 2836-2842, 2001). So, if one protein sample is digested with trypsin in a buffer with O18 water, and another sample is digested in a buffer with normal H₂O, the ratio of these two protein concentrations can be determined from singles of O16/O18 labeled peptides. Since this method does not require any chemical derivation, it provides a simple way to use stable isotopic labeling proteins.

5) Metabolic Labeling

In one method developed by Chait et al (PNAS, 96, 6591-6596, 1999), the cells were grown in two different media, one with normal nitrogen source and other with N15 replaced nitrogen source. So proteins are isotopically labeled with two different nitrogen in its amino acids. In similar method developed by Mann, Leu and/or Lys are replaced by heavy isotopic in a culture media, resulting the heavy isotopic labeling peptides containing these Leu/Lys amino acids. Another pool of samples was grown in normal growth media so the peptides are of normal isotope. The advantage of these metabolic labeling is that there is no post-digestion derivatization needs. The disadvantages include high cost and inability to label tissue samples.

Basis Description of Mass Spectrometry

a) Ionization

In principle, a mass spectrometer can do is to measure gas phase charged particles. So, a mass spectrometer often contains three parts: an ionization source which convert analyte into a gas phase charged particle, a mass analyzer that can determines the m/z of gas phase particles, and a data system that records and analyzes data. For analysis proteins/peptides, two ionization methods are almost widely used nowadays: Matrix-Assisted-Laser-Description/Ionization (MALDI) and Electrospray Ionization (ESI). These two ionization methods are often considered as “soft” ionization methods since they are capable to generate gas phase molecular ions without extensive degradation of heat-labile molecules such as peptides or proteins. Both methods were based on discoveries in late 1980s. The development of these two ionization techniques has dramatically increases the sensitivity in analyzing biological polymers by hundreds or even thousands folds.

In MALDI method, an analyte such as peptides or proteins are imbedded in crystalline matrix of certain weak acids (DHB, alpha-CCA, are two most commonly used as matrix), which are capable to absorb energy from a laser beam. During ionization process, a sample is radiated with a laser beam, and some energy adsorbed by matrix is transferred to become the internal energy of analyte molecules, and lead to vaporization of the intact analyte molecules in charged forms. These gas phase ions are accelerated by an electromagnetic field towards a mass analyzer.

In ESI, a solution sample containing analyte such as proteins or peptides is charged by high-voltage electric field, typically in a range of 500 volt to 2000 volt. As the solution is pumped through a fine needle, a liquid spray of charged droplets are formed. In the process called desolvation, the non-charged solvent molecules in charged droplets are gradually evaporated, which leads to decrease of the diameters of liquid droplets and increase the electric field inside the droplets. At a certain point, the repulsive force of Columbus interaction between charged molecules inside a droplet become so strong that the surface tension of the liquid droplet can no longer hold charged molecules together. So such a droplet will split into many smaller particles. With repeat of this process, the gas phased charged molecules are formed. These charged gas phase molecules are then transfer in mass analyzer for measuring m/z.

Some other ionization methods can also used in mass spectrometry, including electron ionization (EI), chemical ionization (CI), fast atom Bombardment (FAD), or atmospheric pressure chemical ionization (APCI). However, for the analyzing biopolymers such as proteins or peptides, use of these ionization methods is very rare and limited nowadays.

b) Mass Analyzer

The earliest mass analyzer is a magnetic sector, in which a magnetic field is applied in a mass analyzer. A charged ion entering into this magnetic field will be subjected to the force and will travel in a circular motion, with its radius depending upon the m/z of the ion and speed of the ion at a giving magnetic field. Therefore, when a range of magnetic field is scanned in a magnetic sector, ions with different m/z can be separated and recorded by such a magnetic sector machine,

Fourier-transform ion cyclotron resonance (FTICR) is another type of mass analyzer. This type mass analyzer is based on the principle that a charged particle orbiting in a magnetic field can be excited by a radio frequency (RF) signal. The excited ions can then produce an image currant, which can be Fourier-transformed to calculate m/z of the ion. Although FTICR based mass spectrometer is often of high cost, it offers some distinct advantages over other type of mass analyzers: high resolution and high mass accuracy.

A Time-of-Flight (TOF) mass analyzer is widely used mass analyzer these days. Its operation principle is quite simple: when a charged molecule is accelerated in an electric field, the translation energy it has acquired will be determined by the electric field disregarding the mass of the molecule. So ions with different masses will acquire same amount translation energy after accelerated in the same electric field. As a result, ions with different mass will acquire different speed after accelerated by an electric field. When they ions travel into a zero-field path, the time it takes for each ion to reach to a detector will depend on m/z of each ions. So, by recording the time an ion takes to reach to a detector, its m/z can be measured.

Quadrupole is another type of mass analyzer that has been used widely in couple with ESI ion source. A quadrupole is composed of four parallel metal rods, with a direct current (DC) and a radio-frequency (RF) potential applied on it. At certain DC and RF, only ions with a particular m/z can be passed through a quadrupole. So it filters ion according their m/z. By scanning a range of RF, different m/z of ions can be measured. As a mass analyzer, it offers several advantages that make it the best choice for many type of mass spectrometers: 1) it can measure mass at relatively poor vacuum so it reduce the need for a high vacuum system. 2). Three quadruples can be coupled together to make triple quadruple mass analyzer, which can be used not only to measure m/z of an analyte such as peptides, but also the fragments from breakdown of the analyte (tandem mass spectrometry). Such capacity allows a triple quadrupole mass spectrometer to sequence peptides. 3). The lower requirement of the vacuum system plus low cost of quadrupole mass analyzer itself can significantly reduce the cost of a mass spectrometer.

Ion trap can be considered as a subtype of quadrupole mass analyzer. Instead of four parallel metal rods used in a quadrupole mass analyzer, there are two hyperbolic endcap electrodes and one ring electrode used in an ion trap mass analyzer. By applying superimposed DC and RF, ions of certain m/z can be stored inside an ion trap, and ions can also be selectively ejected from an ion trap according to their m/z. So similar to quadrupole mass analyzer, m/z of an analyte can be measured by scanning a range of RF. The main different between ion trap mass analyzer and quadrupole mass analyzer is that ions pass through a quadrupole while can be stored in an ion trap. As a result, ion trap can accumulate ions for relatively long time to achieve higher detection sensitivity. Such advantage, plus the other advantages shared with a quadrupole mass analyzer such as lower vacuum requirement, ms/ms capacity and low cost, make the ESI ion trap mass spectrometer the top choice in protein/peptide sequencing.

Enzymatic Digestion

In order to identify a protein sample, it is often necessary to carry out proteolytic digestion of proteins before mass spectrometric analysis, since peptides with mass range of 500 D-300D can be easily sequenced by a tandem mass spectrometer. Trypsin, with cleavage site at Arg and Lys residues, is often the enzyme of the choice for protein digestion, since peptides from trypsin digestion have a average mass about 1500 Da, and often contain two or three positive charges, which are suitable to generate a ladder of product ions, which can be used to search for peptide sequence information. Other proteolytic enzymes, including Asp-N, Glu-C, Lys-C and Arg-C, can also be used for this purpose under some circumstance (e.g. a protein with very unusual amino acid sequence such as too many or too few Arg and Lys). CNBr has been used widely in internal chemical sequencing (Edman sequencing), but it is rarely used for MS based sequencing due to its high toxicity and the large size of peptides it generated.

Computer Database Search and De Novo Sequencing

With the rapid progress in whole genome sequencing projects, the genomes of many common used experimental organisms have been sequenced with complete or nearly complete coverage. As a result, the task of protein identification become much easier if a protein sample is prepared from a species that its genome has been sequenced, since it is often possible to identify a protein based on MS/MS information of a single peptide.

Such database search is often carried out by a computer with some special software. Several software packages are now commercially available, including Sequest (from Thermo Electron), Mascot (from Matrix Science). There are also many other in-house developed software programs used for MS based protein sequencing. The non-redundant protein database from GenBank is the commonly used protein database. It is compiled from GenBank CDS translations, PIR, SWISS-PROT, PRF, and PDB, and NCBI has made strong efforts to cross-reference the sequences in these databases in order to avoid duplication. The sequence database can be freely downloaded.

In the case that a genome of an organism has not been sequenced, MS based protein identification can also be carried out in one of two ways: 1) based on homology of the conserved sequence across species. For example, if a protein samples is prepared from donkey, its genome has not been sequenced now and only limited number of protein sequences from this species are available, one can search against all the available mammalian genome. There is a high possibility that some peptides from such a donkey protein are identical to its corresponding sequence in other mammalian species. Even though most of the donkey protein sequence is still unknown, some clue can be revealed about the identity of the protein, and the partial sequence information can be used to make probe to clone cDNA of the gene coding for the protein. 2). Some incomplete peptide sequence can be deduced based on MS/MS spectra of a peptide, ether manually or with help of some computer programs. The deduced sequence can be used for homology search to find homologous proteins across species, and can also be used to make DNA oligo probe to clone cDNA of the encoding gene.

While the following preferred embodiments are used to demonstrate the principle of my invention and its potential applications, I wish it understood that I do not intend to be restricted solely thereto, but rather that I do intend to include all embodiments thereof which would be apparent to one skilled in the art and which come within the spirit and scope of my invention.

### EXAMPLES

### Example 1

### Identifying Potential Drug Targets by Affinity Chromatography

Background

The knowledge about the true molecular targets and selectivity of a lead inhibitor is important for lead validation process and for the correct interpretation of its biological and pharmaceutical effect in therapeutic intervention. Various studies indicated that conventional lead validation methods, such as kinase panel based selectivity assay, often draw incorrect or incomplete conclusion about cellular protein targets of a lead compound. Thus, proteome-wide assessment can provide a very valuable approach in the lead validation process. In the following example, we used drug affinity chromatography, isotopic labeling, mass spectrometry and bioinformatic tools to systematically profile the in vivo protein targets of a lead compound.

Diagram of The Experimental Procedure

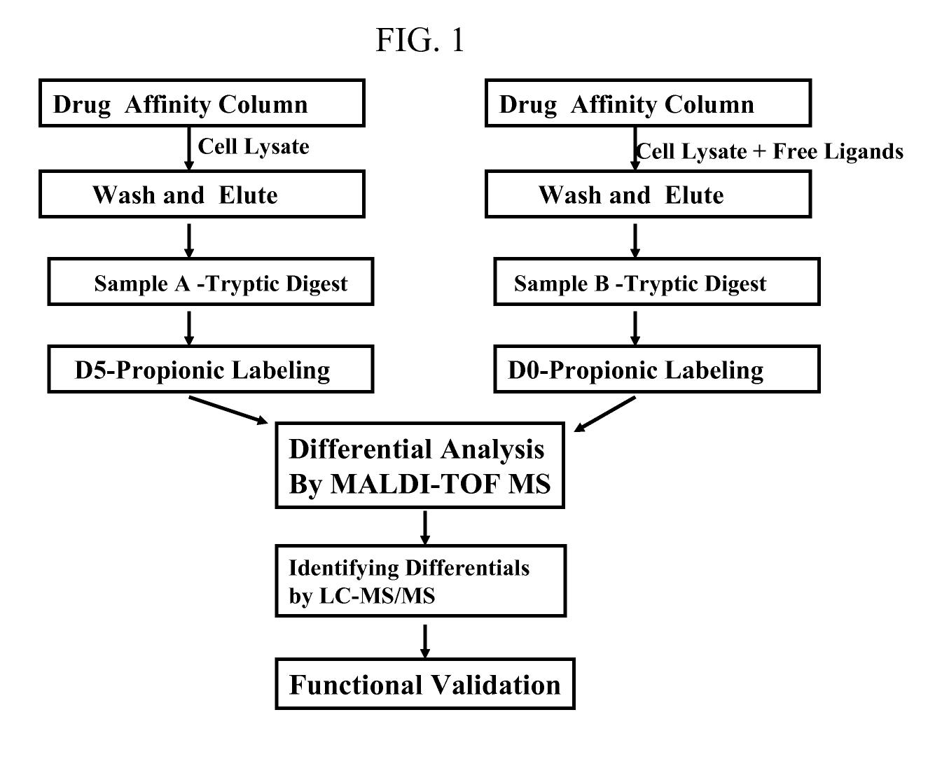

As illustrated in FIG. 1, an affinity purification column is prepared by linking the ligands to a support matrix. Cell lysate is divided into two sets of samples, in one of which the free ligands are added to certain concentration. Both sets of samples are loaded onto the affinity columns and the columns are washed to remove unbound proteins. Bound proteins are eluted, and then subject to trypsin digestion. Peptide mixtures from different set of samples are labeled with different isotope. Labeled peptides from both sets are combined together and analyzed by mass spectrometry.

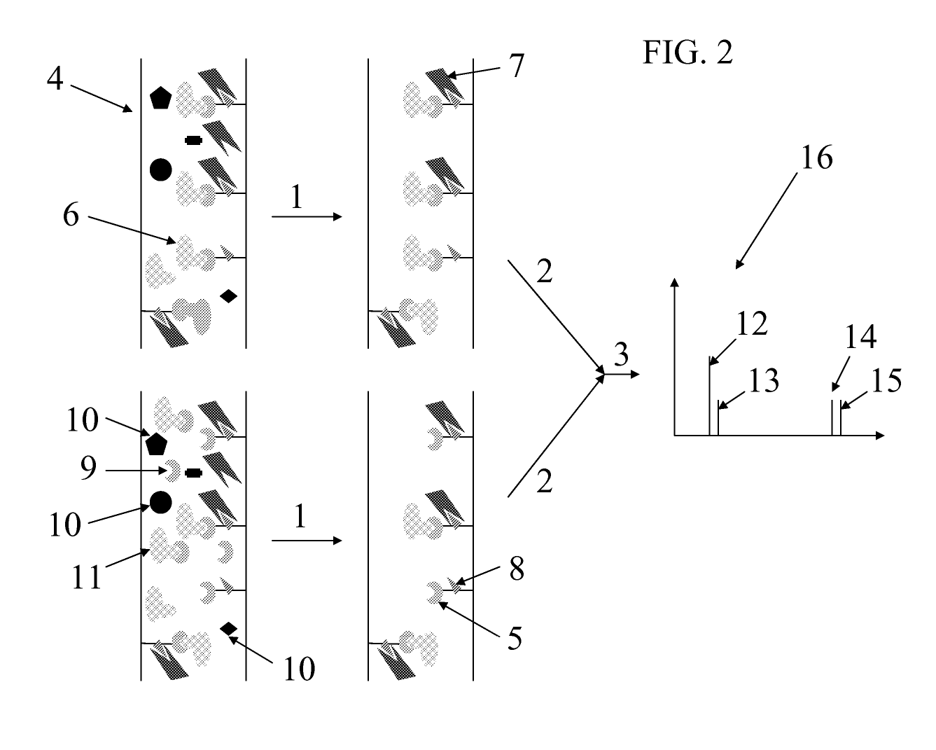

FIG. 2 illustrates the principle of the invention. Cell lysate is divided into two sets of samples. Each set is loaded to an affinity column 4. The column 4 is then washed 1, and bound proteins are eluted, trypsin treated and labeled 2. Labeled peptides are combined together 3, and analyzed by mass spectrometry. The upper panel demonstrates what happens at the affinity purification step in the absence of free ligands 9. The column 4 contains immobilized ligands 5 on a matrix. The target protein 6 is specifically bound to the ligand 5. A contaminant protein 7 is nonspecifically bound to a site 8 in the column 4. Unbound proteins 10 are washed away from the column 4. The target protein 6 and the contaminant protein 7 are represented as peak 12 and peak 14, respectively the on an MS/MS spectrum 16. The lower panel demonstrates what happens at the affinity purification step in the presence of free ligands 9. A portion of target protein 7 is bound to the free ligands 9. Such binding complexes 11 are lost during the wash step 1. As the result, fewer amounts of target proteins 7 are retained on the column 4, and its peak 13 appears smaller on the MS/MS spectrum 16. However, the addition of free ligands 9 does not affect binding of the contaminant protein 7, and its peak 15 is largely unchanged compared to the peak 14 after normalization.

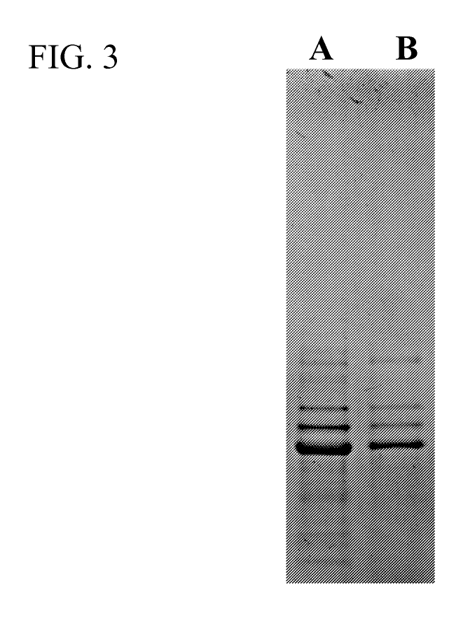

In FIG. 3, Sample A is the elution of proteins from drug affinity purification. Sample B was the control sample, which free ligands were added to cell lysate before affinity purification. All other conditions were same for Sample A and B.

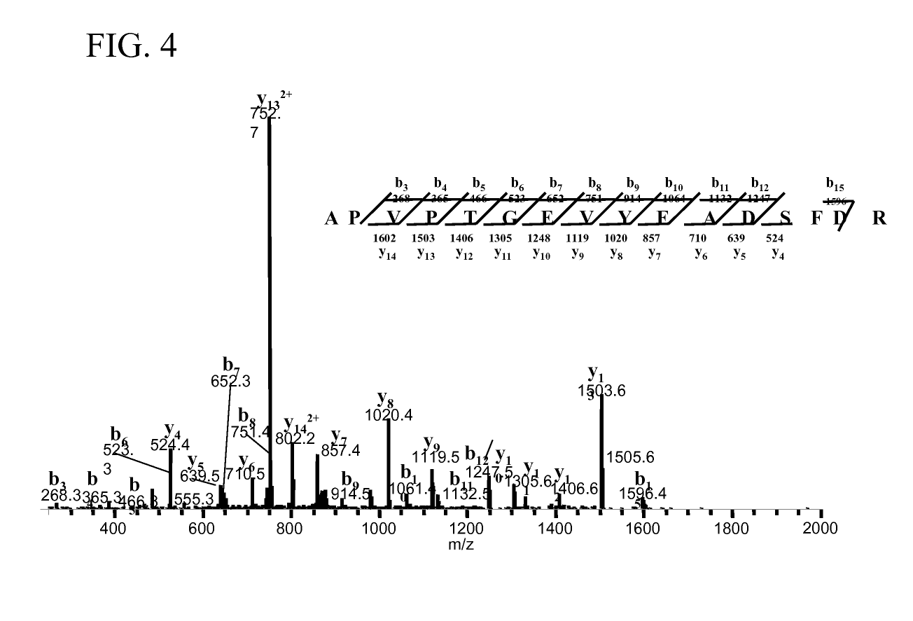

FIG. 4 demonstrates identification of the potential drug targets by LC-MS/MS. MS/MS spectrum obtained from the fragmentation of the precursor ion at m/z 886.6. LC-MS/MS (tandem mass spectrometry) was carried out on the unlabeled peptide mixture using LCQ ion trap mass spectrometer on-line coupled with a Beckman Gold HPLC with a 75 micrometer ID C18 column. The precursor ions were selected based on the corresponding differential peptide identified by MALDI-TOF MS. The peptide sequence identified by bioinformatic analysis and interpretation are reported here. The peptide fragmentation nomenclature is according to one proposed by Roepstorff and Fohlman (P. Roepstorff and J. Fohlman, “Proposal for Common Nomenclature for Sequence Ions in Mass Spectra of Peptides”, Biom. Mass Spectrom. Vol. 11, 601, 1984).

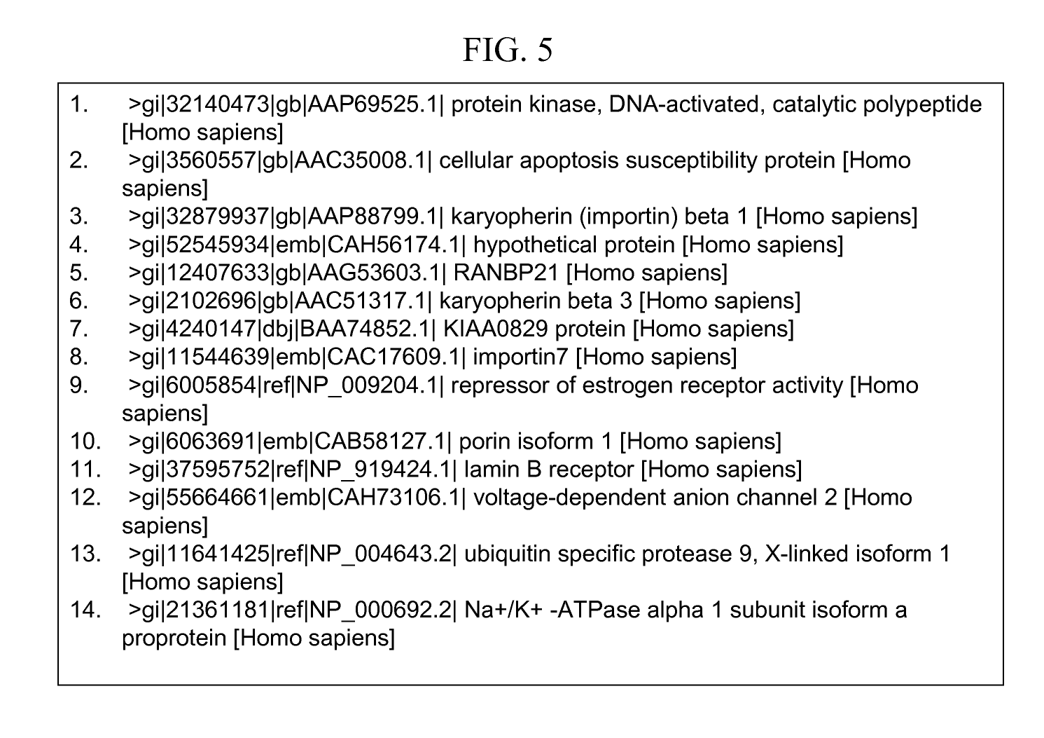

FIG. 5 is a partial list of different proteins identified by searching through GenBank with the mass spectrometry data.

Materials

Sequencing grade, modified trypsin was obtained from Promega (Madison, Wis.). Propionic-d10 was synthesized by Cambridge Isotope Laboratories, Inc (Boston, Mass.) with 98% atom purity. Acetic acid (HOAC) is in Ultrapure grade from J. T. Baker (Pillipsburg, N.J.). Electrophoresis purity urea is from Bio-Rad (Richmond, Calif.). All other reagents, if not specified, were from Sigma-Aldrich (St. Lois, Mo.).

Experimental Procedure

Step 1. Making Drug Affinity Column.

A small molecule lead compound was identified during the high throughput screening against a in vitro target of a receptor protein kinase. An analog of this lead compound, AN1001, which is structurally similar to the lead compound but contains an additional primary amine group at the side that does not involved in its binding to the target protein was used as the ligand to make affinity column. It was demonstrated that the analog had a similar inhibition profile as the lead compound.

To immobilize the ligands, NHS-activated Sepharose™ 4 Fast Flow from Amersham (Catalog Number 17-0981-01) was used. The ligands were coupled to the matrix by covalent attachment of the primary amino group in a ligand to the NHS (N-hydroxysuccinimide) activated group with the 14-atom spacer arm. The matrix was made of highly cross-linked 4% agarose with average bead size of 90 mM. The coupling reaction was carried out according to vendor's recommended protocol. Briefly, 10 ml of NHS-activated Sepharose was washed with 100 ml cold 1 mM HCl. The ligand compound was dissolved in 5 ml 50% acetonitrile in a concentration of 250 mmol/ml. Then, the ligand solution and the matrix were mixed together and 5 ml 0.5 M phosphate buffer (pH7.5) was added to the mix, and the coupling reaction was carried out at room temperature for 4 hrs. Then, 20 ml 1 M Tris buffer (pH 8.5) was added to the reaction mix and incubated at 37 C for 4 hours. This step blocked non-reacted groups on the matrix. The coupled affinity media was then washed with Tris buffer three times before use.

Step 2. Purification of Target Proteins

HEK 293 cells were cultured to 90% confluence in DMEM-F12 containing 10% fetal bovine serum. The cells were harvested and washed twice with PBS. The cells were then lysed in a non-denaturing lysis buffer of 50 mM Tris-HCl (pH 7.4), 300 mM NaCl, 0.9% (v/v) Triton X-100. Protease inhibitors were added as following: 1 mM PMSF, 2 mg/ml leupeptin, and 10 mM iodoacetamide. The cell lysate was clarified by ultracentrifugation and then incubated with affinity beads for 1 hour at 37oC. The slurry was loaded into an empty column and washed exhaustively with PBS containing 0.05% Tween 20. The beads were then washed with PBS without Tween 20. The bound proteins were eluted from the beads with 6 M guanidine hydrochloride containing 2% CHAPS. The eluted proteins were dialyzed against 10 mM ammonium bicarbonate (pH 8.5). This sample was labeled as Sample A.

The control sample was prepared in same procedure as described above, except that the free ligand was added to the cell lysate at the final concentration of 500 mmol/ml before incubated with affinity media. Similarly, the eluted proteins from the control sample were dialyzed against 10 mM ammonium bicarbonate (pH 8.5). This control sample was labeled as Sample B.

Both sample A and B were stored at −80oC. if they were not used immediately.

Step 3. Digestion of Sample A and Sample B.

An aliquot of 10 ml sample A and B were used to run SDS-PAGE. The gel was stained with Coomassie Blue. The gel photo was shown in FIG. 2. The gel can give a good indication about quality of the samples purified from affinity purification. It was used to estimate the amount of proteins in the samples.

Before digestion, both samples were denatured by adding urea to 8M, reduced by DTT, and alkylated by iodoacetamide. DTT was added to sample A and B at the final concentration of 5 mM and Incubated at 65 C for 30 min. Then, iodoacetamide was added to both samples at final concentration of 10 mM, and incubated in dark for 60 minutes. The reaction mixtures were then diluted with 50 mM ammonium bicarbonate (pH 8.5) to 1 M urea, and then Promega sequencing grade modified trypsin was added to about 1/10 of total protein amount. The reaction mixes were incubated at 37 C for 12 hours.

Step 4. Isotopic Labeling of Peptide Pools from Trypsin Digestion of Sample A and B.

The two samples from trypsin digestion of Sample A and B were used differentially labeled following the NIT procedure (Rapid Communication in Mass Spectrometry, 16: 2325-2332, 2002). Both peptide mixtures were dissolved in 50 mM CAPS buffer (pH 10.7) and O-methylisourea was added to final concentration 100 mM. The reactions were carried out at room temperature for 18 hours. The completion of this guanidination reaction was checked by MALDI-TOF MS. Then, peptides sample from Sample A digestion was acylated by D10-propionic anhydride, and peptide sample from Sample B (control) digest was acylated by D0-propionic anhydride, where D10 refers to a molecule where all ten hydrogen atoms have been replaced by deuterium, and D0 refers to a molecule where no deuterium have been incorporated in place of the hydrogen. Due to the hydrolysis of the anhydride in aqueous solutions, the mole amount of propionic anhydride was in >100 fold excess to that of peptides. Since the buffer for guanidination reaction is compatible with acylation reaction, propionic anhydride (D10 or D0) was added directly to the guanidination reaction mixture. The reactions were allowed to proceed at room temperature for 4 hours to reach completeness. Two reaction mixtures were then combined into a 1.5 ml microtube, and hydroxylamine was added to a final concentration of 1 M. This step is necessary to remove any esterification of tyrosine residues. The reaction was complete within one hour at room temperature. The combined sample was then desalted by using Zeba Desalt Spin Column (Pierce, Catalog No. 89882). The vendor recommended desalting procedure was used.

About 80% of samples from the trypsin digests of Sample A and B were used in this isotopic labeling step. Remaining 20% trypsin digestion samples were saved for later use. Since NIT labeling neutralizes the positive charge at peptide N-terminals, labeled peptides may become singly charged under LC-MS/MS condition. As a result, some big peptide could be out of the mass range for MS/MS by LCQ mass spectrometer (0-2000 Da), or its MS/MS spectra may become more difficult to assign to a peptide with high confidence. Using unlabeled peptide to further confirm the identification of protein differentials may make identifications easier and more reliable.

Step 5. Fractionation of Peptide Pools by SCX.

Since SDS-PAGE of Sample A and Sample B indicated that there were quite a large number of proteins present in both samples even before trypsin digestions, it was clear to us that fractionation of the combined isotopically labeled peptides mixture before mass spectrometric readout could significantly increase the chance in identifying differential peptides by MALDI-TOF MS. The fractionation was carried out with strong cation exchange (SCX) chromatography. A 2.1 mm×20 cm polysulfoethyl A column (PolyLC, Inc., Columbia, Md.) connected to a Beckman Gold HPLC was used. A 40 minute gradient from 5% solvent B to 50% Solvent B was used, and each fraction was collected in a 2-minutes period. Solvent A: 5 mM phosphate buffer, 25% ACN, pH3.0; Solvent B: 5 mM phosphate buffer, 25% ACN, 0.5 M NaCl, pH3.0. Totally twenty fractions were collected.

Step 6. Differential Analysis by MALDI-TOF Mass Spectrometry

Each fraction from SCX was desalted by using PepClean C18 Spin Column (Pierce, Catalog No. 89870). The peptides were eluted with 50 ml 75% acetonitrile. The elutions were then dried down in a SpeedVac to about 5 ml. 1 ml of it from each fraction was mixed with 1 ml matrix solution of alpha-cyano-4-hydroxycinnamic acid (HCCA) and loaded on a MALDI probe. A ProteomeWork System reflectron mass spectrometer (Micromass, UK) was used for acquiring MALDI-TOF data. MALDI-TOF spectra were calibrated by internal calibration using two standard peptides doped in the HCCA matrix. The MALDI-TOF data was processed by an in-house developed software tool. Due to isotopic labeling of peptide N-terminals, most peptides show a doublet of 5 Da apart, with the normalized intensities of 1:1 ratio (10% error range). The differential peptides were identified if the normalized intensities of two peaks in a doublet have a difference large than 50% of the average peak intensity. These peaks were subjected to LC-MS/MS analysis described in the following.

Step 7. Identifying the Potential Drug Target Proteins by LC-MS/MS

The differential peptides identified by MALDI-TOF MS were then sequenced by LC-MS/MS to identify differential proteins. An electrospray ion trap mass spectrometer (LCQ, Finnigan MAT, San Jose, Calif.) coupled on-line with a Beckman Gold HPLC system with a 75 mm ID×10 cm length column was used for LC-MS/MS. The column was packed with 3 mM reverse-phase C18 beads. Mobile phases used were as following: Solvent A (2% ACN, 97.9% H₂O, 0.1% Formic Acid); Solvent B (90% ACN, 9.9% H₂O, 0.1% Formic acid). A linear gradient from 2% B to 90% B in 60 minutes was used for the LC/MS/MS experiment. The data was acquired by Xcalibur (Thermo) software in a data-dependent mode, in which a list of parent ions was preset, based on the massed of the differential peptides identified by MALDI-TOF MS in each fraction. The Automatic Gain Control (AGC) function of the mass spectrometer was turned on to prevent the repetitive MS/MS experiment of same parent ion. The raw data was searched against NCBI non-redundant protein database using in-house developed software tools.

In the case that the amino acid sequence of a differential peptide could not be non-equivocally assigned based on the MS/MS spectrum of the NIT labeled peptide, effort was made to generate MS/MS spectrum of its corresponding non-labeled peptide, in almost all the cased, those differential peptides can then be successfully assigned by using non-labeled samples.

An example MS/MS spectrum and its assignment are shown in FIG. 3.

Results and Discussion

A large number of proteins were identified from Sample A and Sample B (control). We identified 137 proteins in Sample A, and 117 proteins in Sample B. About 24 proteins are presence in Sample A but are absent or >50% reduced in Sample B. Some of these differential proteins are listed in the Table 1. These proteins identified here were subjected to further validation by functional studied.

Conclusion

We demonstrated here that combining drug affinity chromatography with proteomics and bioinformatic technologies can provide a very powerful method for profiling cellular targets of a lead compound. By using Nano-LC-MS/MS based protein identification and bioinformatics based data mining, it is possible to identify proteins that specifically bind to lead inhibitors, even though the non-specific binds by molecular chaperons or other abundant cellular proteins are still present. With this approach, it is no longer required to purify target proteins to homogeneity or as a distinctive gel band, and it is possible to obtain more complete information about true cellular targets of a lead compound.

### Example 2

Sometimes it is desirable to identify those unwanted binding proteins. For instance, the ligand contains multiple potential binding sites, and it is hard to obtain a free analog that consists of the interested binding site only. We could use free analogs resembling other binding sites of the ligand to identify those unwanted binding proteins.

### Example 3

This invention can be used to determine the binding affinity of a ligand to its target proteins. In this case, the initial sample is divided into several sets, each of which contains different concentrations of free ligands. By comparing with the one without free ligand addition, a plot can be drawn with “amount of target proteins retained on column” vs. “concentration of free ligand added.” The binding affinity of the ligand to its target proteins can be calculated accordingly.

### Example 4

This invention can be used to compare the binding affinity of different ligands to commonly shared target proteins. A group of analogs can all bind to many target proteins. However, different analog might have different binding affinity to each target. It is useful to determine the preference of different ligand to each target protein.

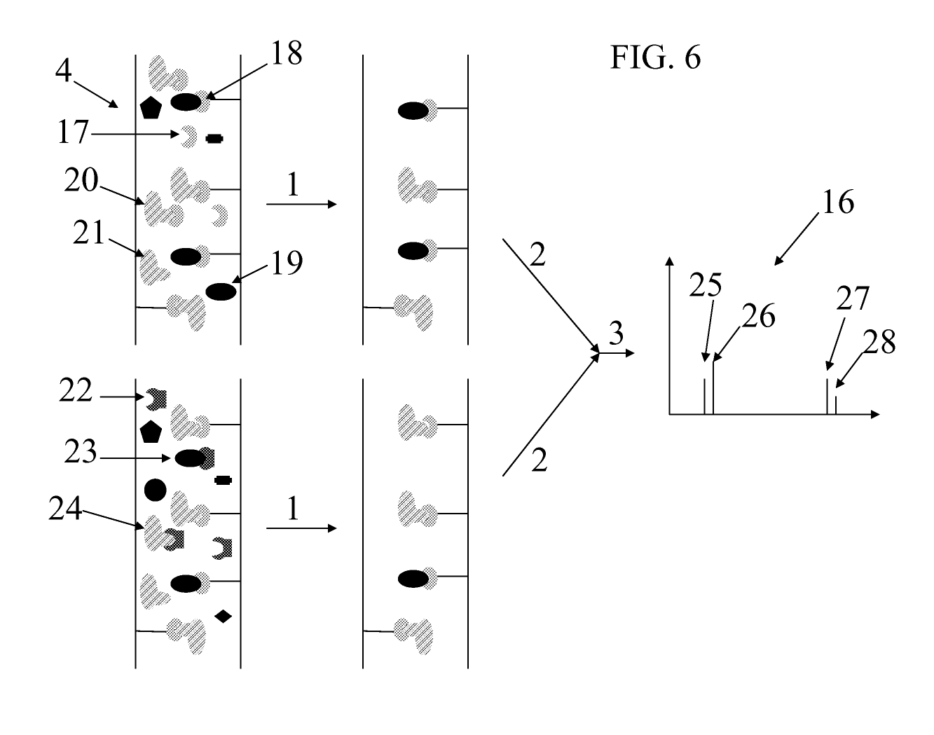

FIG. 6 illustrates the principle of the invention in comparing the binding affinity of different ligands to commonly shared target proteins. Cell lysate is divided into two sets of samples. Each set is loaded to an affinity column 4. Both target proteins 21, 19 could bind to immobilized ligands 18. The column 4 is then washed 1, and bound proteins are eluted, trypsin treated and labeled 2. Labeled peptides are combined together 3, and analyzed by mass spectrometry. The upper panel demonstrates what happens at the affinity purification step in the presence of the first free ligands 17, which has higher binding affinity to the target protein 21. Therefore, addition of free ligand 17 has more negative impact on the binding of target protein 21 to the column. The target protein 21 and 19 are represented as peak 25 and peak 27, respectively the on an MS/MS spectrum 16. The lower panel demonstrates what happens at the affinity purification step in the presence of the second free ligands 22, which has higher binding affinity to the target protein 19. Therefore, addition of free ligand 22 has more negative impact on the binding of target protein 19 to the column. The target protein 21 and 19 are represented as peak 26 and peak 28, respectively the on an MS/MS spectrum 16. By comparing the amount of target proteins retained on the column in the presence of different free ligands, we could determine the preference of different ligands to different target proteins.

### Example 5

This invention can be used to compare the binding affinity of different target proteins to a ligand. A lead compound is often generated from HTS with a target protein. The lead compound is then used to identify other proteins that it binds to. Naturally, a question is whether the newly discovered target proteins binds stronger to the lead compared to the original target. In this case, we could use purified original target as free ligand and determine to what extend the original target protein could replace the newly discovered target proteins on an affinity column.

### Example 6

This invention can also be used to determine indirect effect of a factor on the binding of a ligand to its target proteins. For instance, many proteins have multiple binding sites for different ligands, and binding of a ligand at one site might affect its ability to bind to other ligand at another site. To determine effect of a ligand A on the binding of a ligand B to the target protein, we could immobilize ligand B on an affinity column and use ligand A as free ligand.

What is claimed is:

1. A method for identifying protein targets, which interact with a chemical or biological compound, from an initial sample containing a plurality of proteins, comprising:
a. a binding matrix generated by immobilizing said chemical compound on a supporting substance;
b. at least two sets of experimental samples derived from said initial sample, wherein at least one set of said experimental samples contains at least one chasing factor that is missing or is present at a different concentration in other set(s), wherein said chasing factor can directly or indirectly either compete with said binding matrix to interact with said protein targets or compete with said protein targets to interact with said binding matrix;
c. a purification means to purify proteins from each set of said experimental samples, by retaining said proteins with said binding matrix under a first condition, and then releasing said proteins from said binding matrix under a second condition;
d. a labeling means to attach different isotopic variant of a chemical moiety to said proteins purified from different set of said experimental samples to yield different labeled pools of said proteins;
e. a means to combine at least two said labeled pools according to a defined ratio to yield a mixture; and
said mixture is then subject to mass analysis, where a significant peptide is identified based upon its mass spectrometric signal differentiation between said pool A2 and said pool B2 after normalization, and the protein from which said peptide is derived is then identified.

2. A method as in claim 1, wherein said chemical compound is a drug or drug candidate.

3. A method as in claim 1, wherein said supporting substance is a form of agarose beads.

4. A method as in claim 1, wherein said chasing factor is identical to said chemical compound or an analog of said chemical compound.

5. A method as in claim 1, wherein said chasing factor is a protein.

6. A method as in claim 1, wherein said initial sample is derived from a cell extract.

7. A method as in claim 1, further comprising a digestion means to digest said protein targets purified from different sets of said experimental samples with at least one proteolytic enzyme, performed either before or after said labeling means.

8. A method as in claim 7, wherein said chemical compound or biological is a drug or drug candidate.

9. A method as in claim 7, wherein said supporting substance is a form of agarose beads.

10. A method as in claim 7, wherein said chasing factor is identical to said chemical or biological compound or an analog of said chemical or biological compound.

11. A method as in claim 7, wherein said chasing factor is a protein.

12. A method as in claim 7, wherein said initial sample is derived from a cell extract.

13. A method for identifying proteins, which specifically interact with a first chemical or biological compound, comprising:
a. immobilizing said chemical or biological compound on a supporting substance to form an affinity matrix;
b. dividing an initial sample containing a plurality of proteins into at least two sets, namely sample A and sample B, wherein said sample B contains at least one second chemical or biological compound, which distinguishes said sample B from said sample A;
c. processing said sample A as following:
(1) contacting said sample A with said affinity matrix;
(2) isolating proteins retained by said affinity matrix;
(3) digesting said proteins isolated from said affinity matrix with at least one proteolytic enzyme to generate a first peptide pool A1;
(4) labeling said peptide pool A1 with a first isotopic variant of a chemical moiety to yield a first isotope-labeled peptide pool A2;
d. processing said sample B as following:
(1) contacting said sample B with said affinity matrix;
(2) isolating proteins retained by said affinity matrix;
(3) digesting said proteins isolated from said affinity matrix with at least one proteolytic enzyme to generate a second peptide pool B1;
(4) labeling said peptide pool B1 with a second isotopic variant of said chemical moiety to yield a second isotope-labeled peptide pool B2;
e. combining a portion of said peptide pool A2 and a portion of said peptide pool B2 together to yield a combined peptide sample; and
f. subjecting said combined peptide sample to mass spectrometric analysis, where a significant peptide is identified based upon its mass spectrometric signal differentiation between said first isotope-labeling and said second isotope-labeling after normalization, and the protein from which said peptide is derived is then identified.

14. A method as in claim 13, wherein said first chemical or biological compound is a drug or drug candidate.

15. A method as in claim 13, wherein said supporting substance is a form of agarose beads.

16. A method as in claim 13, wherein said second chemical or biological compound is identical with said first chemical or biological compound or an analog of said first chemical or biological compound.

17. A method as in claim 13, wherein said initial sample is derived from a cell extract.

18. A method for identifying proteins, which specifically interact with a first chemical or biological compound or biological, comprising:
a. immobilizing said chemical or biological compound on a supporting substance to form an affinity matrix;
b. dividing an initial sample containing a plurality of proteins into at least two sets, namely sample A and sample B, wherein said sample B contains at least one second chemical or biological compound, which distinguishes said sample B from said sample A;
c. processing said sample A as following:
(1) contacting said sample A with said affinity matrix;
(2) isolating proteins retained by said affinity matrix;
(3) attaching said proteins isolated from said affinity matrix with a first isotopic variant of a chemical moiety to yield a first isotope-labeled peptide pool A1;
d. processing said sample B as following:
(1) contacting said sample B with said affinity matrix;
(2) isolating proteins retained by said affinity matrix;
(3) attaching said proteins isolated from said affinity matrix with a second isotopic variant of said chemical moiety to yield a second isotope-labeled peptide pool B1;
e. combining a portion of said pool A1 and said pool B1 to yield a combined protein sample;
f. digesting said combined protein sample with at least one proteolytic enzymes to yield a combined peptide sample;
g. subjecting said combined peptide sample to mass spectrometric analysis, where a significant peptide is identified based upon its mass spectrometric signal differentiation between said first isotope-labeling and said second isotope-labeling after normalization, and the protein from which said peptide is derived is then identified.

19. A method as in claim 18, wherein said first chemical or biological compound is a drug or drug candidate.

20. A method as in claim 18, wherein said supporting substance is a form of agarose beads.

21. A method as in claim 18, wherein said second chemical or biological compound is identical with said first chemical or biological compound or an analog of said first chemical or biological compound.

22. A method as in claim 18, wherein said initial sample is derived from a cell extract.

23. A method for identifying proteins, which specifically interact with a first chemical or biological compound, comprising:
a. immobilizing said chemical or biological compound on a supporting substance to form an affinity matrix;
b. dividing an initial sample containing a plurality of proteins into at least two sets, namely sample A and sample B, wherein said sample B contains at least one second chemical or biological compound, which distinguishes said sample B from said sample A;
c. processing said sample A as following:
(1) contacting said sample A with said affinity matrix;
(2) isolating proteins retained by said affinity matrix;
(3) digesting said protein isolated from said affinity matrix with at least one proteolytic enzyme in a solution containing a chemical that can attach a first isotopic variant of a chemical moiety to digested peptides to yield a first isotope-labeled peptide pool A1;
d. processing said sample B as following:
(1) contacting said sample B with said affinity matrix;
(2) isolating proteins retained by said affinity matrix;
(3) digesting said proteins isolated from said affinity matrix with at least one proteolytic enzyme in a solution containing a chemical that can attach a second isotopic variant of said chemical moiety to digested peptides to yield a second isotope-labeled peptide pool B1;
e. combining a portion of said pool A1 and said pool B1 to yield a combined peptide sample;
f. subjecting said combined peptide sample to mass spectrometric analysis, where a significant peptide is identified based upon its mass spectrometric signal differentiation between said first isotope-labeling and said second isotope-labeling after normalization, and the protein from which said peptide is derived is then identified.

24. A method as in claim 23, wherein said first chemical or biological compound is a drug or drug candidate.

25. A method as in claim 23, wherein said supporting substance is a form of agarose beads.

26. A method as in claim 23, wherein said second chemical or biological compound is identical with said first chemical or biological compound or an analog of said first chemical or biological compound.

27. A method as in claim 23, wherein said initial sample is derived from a cell extract.
